(19) United States
(12) Patent Application Publication
(10) Pub. No.: US 2024/0239910 A1
(43) Pub. Date: Jul. 18, 2024

(54) BI-SPECIFIC ACTIVATORS FOR TUMOR THERAPY

(71) Applicant: MEMORIAL SLOAN KETTERING CANCER CENTER, New York, NY (US)

(72) Inventors: Danny Nejad Khalil, New York, NY (US); Jedd D. Wolchok, New York, NY (US); Taha Merghoub, Jersey City, NJ (US)

(21) Appl. No.: 18/472,418

(22) Filed: Sep. 22, 2023

Related U.S. Application Data
(63) Continuation of application No. 16/347,202, filed on May 3, 2019, now abandoned.
(60) Provisional application No. 62/417,706, filed on Nov. 4, 2016.

(51) Int. Cl. C07K 16/28; A61K 9/51; A61P 35/00; C07K 16/30; A61K 39/00

(52) U.S. Cl. CPC C07K 16/2878; A61K 9/5161; A61P 35/00; C07K 16/2818; C07K 16/2866; C07K 16/3053; A61K 2039/507; A61K 2039/54

(57) ABSTRACT

The present invention provides various compositions and methods useful for the treatment of cancer, including, but not limited to, cancers that are resistant to immune checkpoint blockade and/or are resistant to treatment with PD-1, PD-L1 or CTLA-4 inhibitors. In some embodiments the present invention provides compositions comprising “bi-specific activators”—which are nanoparticles having both a CD40 agonist antibody and an antibody specific for a tumor-associated antigen on their surface. In some embodiments such nanoparticles comprise one or more vaccine adjuvants, for example inside the nanoparticles. The present invention also relates to the use of such compositions in the treatment of tumors.

### CROSS-REFERENCE TO RELATED APPLICATIONS

This application is a continuation of U.S. patent application Ser. No. 16/347,202 filed on May 3, 2019, which was a U.S. national stage entry of International Patent Application No. PCT/US17/60064 filed on Nov. 4, 2017, which claimed the benefit of priority of U.S. Provisional Patent Application No. 62/417,706 filed on Nov. 4, 2016, the content of which is hereby incorporated by reference in its entirety.

### INCORPORATION BY REFERENCE

For the purpose of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND

Immune checkpoint blockade (ICB) is an approach to treating cancer that involves blocking inhibitory immune-cell receptors, such as PD-1, PD-L1, and/or CTLA-4, present on T-cells. Several such immune checkpoint inhibitors are currently in use clinically—including pembrolizumab, nivolumab, atezolizumab, and ipilimumab. While such methods can lead to durable and occasionally complete tumor regression in some patients, other patients remain insensitive to such treatments. For example, response rates to anti-PD-1 monotherapy range from approximately 44% in melanoma patients to markedly lower rates in breast and colorectal cancer patients. Accordingly, there is a need in the art for new and improved tumor treatment regimens, including treatments that can be used to treat tumors in that subset of patients for which immune checkpoint inhibitors are not effective.

### SUMMARY OF THE INVENTION

The present invention is based, in part, on a series of important discoveries that are described in more detail in the Examples section of this patent specification. For example, it has now been discovered that certain “bi-specific activator” agents can be used to successfully treat tumors that were previously resistant to treatment with immune checkpoint inhibitors—leading to tumor regression. It is believed that such agents may also be effective in other situations also—for example in the treatment of tumors that are not necessarily resistant to treatment with immune checkpoint inhibitors. In some embodiments such “bi-specific activator” agents may be used alone, while in other embodiments the “bi-specific activator” agents may be used combination with immune modulators (including, for example, immune checkpoint inhibitors or immune activators). Building on these discoveries, and other discoveries presented herein, the present invention provides a variety of new and improved compositions and methods for the treatment of tumors. Some of the main aspects of the present invention are summarized below. Additional aspects of the invention are provided and described in the Detailed Description, Drawings, Examples, and Claims sections of this patent application.

In some embodiments the present invention provides compositions comprising nanoparticles, that comprise a CD40 agonist antibody (for example to engage and activate antigen presenting cells or “APCs”), and an antibody specific for a tumor associated antigen or “TAA” (to engage tumor cells) on the surface of the nanoparticles. Such nanoparticles may be referred to herein as “bi-specific activators”—i.e. comprising both a CD40 agonist antibody and an antibody specific for a TAA. In some embodiments such compositions also comprise one or more agents as “cargo” inside the nanoparticles that can further activate APCs, such as one or more vaccine adjuvants (including, but not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, BCG (bacille Calmette-Guerin) and Corynebacterium parvum) or TLR agonists (including, but not limited to, the TLR4 agonist monophosphoryl lipid A (“MPL”) and/or the TLR3 agonist polyI:C). In some embodiments such compositions optionally also comprise one or more additional agents on the surface of the nanoparticles, such as additional antibodies (e.g. an IL10 receptor blocking antibody or an IL10 blocking antibody). The present invention also provides methods of treating tumors by administering such compositions to subjects in need thereof.

In some such embodiments the nanoparticle is made using any suitable nanoparticle chemistry or technology known in the art. In some such embodiments the nanoparticle comprises one or more agents selected from the group consisting of mannose, chitosan, manosylated chitosan, protamine, chitosan with protamine, albumin, PLGA, and fucoidan. In some such embodiments the nanoparticles are formulated to release any active agents within them (i.e. their cargo) at endosomal pH, for example at the pH of early endosomes. The pH sensitivity of the nanoparticles can be adjusted (e.g., by adjusting their density) so the nanoparticles can be made to degrade within the acidic endosomes of APCs. In some embodiments the chemical features or physical properties (e.g., size, charge, etc) of the nanoparticles can be controlled such that systemic administration will lead to enrichment of the nanoparticles in certain organs of interest (e.g., the liver in the case of tumors within the liver or the lung in the case of tumors within the lungs). Means for altering the chemical or physical properties of nanoparticles to allow for tissue-specific enrichment are known in the art and can be used in connection with the present invention. For example, it is known that galactosamine-modified polymers can be used to target asiolaglycoprotein-receptor overexpressed by liver cells as a means for targeted delivery to the liver. See Seymour et al., “Hepatic drug targeting: phase I evaluation of polymer-bound doxorubicin,” J. Clin. Oncol. 2002, Vol. 20(6), pp. 1668-76, the contents of which are hereby incorporated by reference.

In some embodiments the CD40 agonist antibody used in the methods and compositions described herein is selected from the group consisting of the following antibodies: FGK45, CP-870,984, APX005M, dacetuzumab, and ChiLob 7/4. Other suitable CD40 agonist antibodies are described in WO2005/063289 and WO2013/034904.

In some embodiments the antibody that is specific for a TAA is one that binds to an extracellular tumor protein, or is one that binds to a peptide, or peptide-MHC complex, derived from a tumor protein that is displayed on the surface of a cell in complex with a MHC molecule (i.e. that binds to a peptide fragment presented withing MHC-I or MHC-II, or that binds to that peptide together with the complexed MHC molecule). In some embodiments the TAA is selected from the group consisting of gp75/TRP1 (the antigen target of the antibody TA99), her2, muc1, muc16, CD19, CD20, CD38, SLAMF7, WT1, NY-ESO1, EGFRvIII, tyrosinase, gp100/pmel, Melan-A/MART-1, and TRP2. In some embodiments the TAA is melanoma-associated antigen, such as, for example, a melanoma-associated antigen selected from the group consisting of tyrosinase, gp100/pmel, Melan-A/MART-1, gp75/TRP1, and TRP2. In some embodiments the TAA is selected from the group of tumor antigens described in Cheever et al., “The Prioritization of Cancer Antigens: A National Cancer Institute Pilot Project for the Acceleration of Translational Research,” Clinical Cancer Research, 2009, Vol. 15(17), pp. 5323-5337, the contents of which are hereby incorporated by reference.

In some embodiments the TLR agonist used in the methods and compositions described herein is any TLR agonist known in the art that binds to a TLR expressed by antigen presenting cells (APCs), such as a dendritic cells (DCs), macrophages, tissue-resident macrophages, monocytes, monocyte-derived cells, B-Cells, neutrophils, langerhans cells, histiocytes, or any so-called professional or non-professional APC. In some embodiments the TLR agonist is a TLR4 agonist, such as monophosphoryl lipid A (MPL). In some embodiments the TLR agonist is a TLR3 agonist, such as polyI:C.

In some embodiments the IL10 receptor blocking antibody used in the methods and compositions described herein is the antibody 1B1.3A.

The compositions of the invention may be delivered using any suitable route of administration—whether local or systemic. Suitable routes of local administration include, but are not limited to, intratumoral, intrahepatic, intrapleural, intraocular, intraperitoneal, and intrathecal administration. In preferred embodiments intravenous administration is used. In particular, it has been found that the nanoparticle compositions of the invention are particularly potent when administered intravenously, such that the nanoparticles can be administered intravenously at approximately the same (low) dose with which they are administered intratumorally.

In some embodiments the compositions of the invention may be co-administered with, or otherwise used in a treatment regimen that comprises administration of, an immune checkpoint inhibitor (such as an anti-PD-1, anti-PD-L1, or anti-CTLA-4 agent). In some such embodiments the immune checkpoint inhibitor is administered systemically. However, in other embodiments the immune checkpoint inhibitor is administered locally, such as intratumorally. In some embodiments the immune checkpoint inhibitor (including but not limited to PD-1, PD-L1, and/or CTLA-4 inhibitor) used in the methods and compositions described herein is an antibody. In some such embodiments the immune checkpoint inhibitor is an antibody selected from the group consisting of pembrolizumab, nivolumab, atezolizumab, ipilimumab, and the PD-1 inhibitor antibody RMP1-14.

In some embodiments the subject has any solid tumor, including, but not limited to, a melanoma, a breast tumor, a lung tumor (such as a small cell lung cancer tumor), a prostate tumor, an ovarian tumor, a sarcoma, and a colon tumor. In some embodiments the subject has melanoma and the bi-specific activator comprises an antibody that is specific for a melanoma-associated antigen, such as a melanoma antigen selected from the group consisting of tyrosinase, gp100/pmel, Melan-A/MART-1, gp75/TRP1, and TRP2.

In some such embodiments the subject has a tumor that is resistant to treatment with an immune checkpoint inhibitor. In some such embodiments the subject has a PD-1, PD-L1, and/or CTLA-4 inhibitor resistant tumor. In some such embodiments the subject has previously been treated with an immune checkpoint inhibitor (such as a PD-1, PD-L1, or CTLA-4 inhibitor). In some such embodiments the patient has not previously been treated (with immunotherapy, checkpoint blockade, or otherwise).

These and other embodiments are further described in other sections of this patent application. Furthermore, one of skill in the art will recognize that the various embodiments of the present invention described can be combined in various different ways, and that such combinations are within the scope of the present invention.

### BRIEF DESCRIPTION OF THE DRAWINGS

FIG. 1. Schematic illustration of a treatment regimen used in performing experiments described in several of the Examples. By injecting only one of two tumors throughout the course of the experiment it is possible to separate the effect of the injected tumor from the “abscopal” effect on the distant non-injected tumor. Once treatment begins, tumors are measured twice weekly for at least 90 days.

FIG. 2. Schematic illustration of exemplary “bi-specific activator” nanoparticles having two different antibodies in their surface—CD40 agonist mAbs (to engage and activate APCs) and antibodies specific for tumor cell antigens (to engage tumor cells). The nanoparticles may carry internal cargo (as shown) to further activate the APCs.

FIG. 3A-F. Tumor growth curves of “injected” (FIG. 3A and FIG. 3C) and “non-injected” (FIG. 3B and FIG. 3D) tumors in mice treated with either the “non-formulated mixture” (FIG. 3A and FIG. 3B) and or the bi-specific activator composition (“BiAc”) (FIG. 3C and FIG. 3D)—as further described in Example 2. In FIGS. 3A-3D each line/curve represents measurements of tumor size from one individual tumor over time, with time in days indicated on the X axes, and tumor size (surface area) in mm2 indicated on the Y axes. FIGS. 3 E-F. Average tumor growth curves for “injected” (FIG. 3E) and “non-injected” (FIG. 3F) tumors (depicted individually in FIGS. 3A-D). In FIG. 3E and FIG. 3F diamonds are data points from the “bi-specific activator” (BiAc) treatment group and triangles are data points from the “non-formulated mixture” treatment group—as further described in Example—with time in days indicated on the X axes and tumor size in mm² indicated on the Y axes. Both individual (FIGS. 3A-D) and averaged (FIG. 3E & FIG. 3F) tumor growth curves demonstrate rapid cell-kill of the injected tumor followed by control or eradication of non-injected tumors. In all cases, tumor control was superior with the bi-specific activator.

FIG. 4. provides survival plots for C57BL/6 mice bearing bilateral syngeneic B16 (melanoma) tumors treated using one of the four treatment regimens indicated in the key. Animals were treated as they were for the experiments whose results are shown in FIG. 3 (see Examples 1 and 2). These data demonstrate that animals treated with the bi-specific activator (“BiAc”) intratumorally (“IT”) together with anti-PD-1 therapy intraperitoneally (“IP”) have superior survival, and a superior cure rate, as compared to controls treated with either an antibody isotype control (“isotype”), PD-1 monotherapy (IP), or the non-formulated mixture (IT) at doses equivalent to those used for the bi-specific activator plus anti-PD-1 (IP).

### DETAILED DESCRIPTION

While some of the main embodiments of the present invention are described in the above Summary of the Invention section of this patent application, as well as in the Examples section of this application, this Detailed Description section provides certain additional description relating to the compositions and methods of the present invention, and is intended to be read in conjunction with all other sections of the present patent application.

### Definitions and Abbreviations

As used herein the abbreviation “APC” refers to an Antigen Presenting Cell.

As used herein the abbreviation “CD40” refers to a cluster of differentiation 40—a receptor that may be found on APCs and other cells including tumor cells.

As used herein the abbreviation “DC” refers to a Dendritic Cell

As used herein the abbreviation “IL10” refers to interleukin 10.

As used herein the abbreviation “IL10R” refers to an IL10 receptor, such as an IL10R present on APCs. The term “IL10R” include any and all subunits of the IL10 receptor, including, but not limited to, IL10RA, IL10RB, IL10R1, and IL10R2.

As used herein the abbreviation “IP” refers to intraperitoneal.

As used herein the abbreviation “IT: refers to intratumoral. For example, a drug injected directly into a tumor is delivered intratumorally.

As used herein the abbreviation “IV” refers to intravenous. It is common to administer agents to mice via an IP route, which is considered to be analogous to administering an agent to a human subject by a IV route.

As used herein the abbreviation “MPL” refers to monophosphoryl lipid A. MPL is a TLR4 agonist.

As used herein the abbreviation PD-1″ refers to Programmed Death 1, which is also known as Programmed Death Protein 1 or Programmed Cell Death Protein 1.

As used herein the abbreviation PD-L1 refers to a ligand for PD-1.

As used herein the abbreviation “TLR” refers to Toll-like receptor(s). TLRs on APCs are involved in stimulating APC activation.

As used herein the terms “inhibiting” and “blocking” are used interchangeably, as are the terms “inhibit” or “block” and the terms “inhibitor” or “blocker.”

As used herein, the terms “about” and “approximately,” when used in relation to numerical values, mean within + or −20% of the stated value. Other terms are defined elsewhere in this patent specification, or else are used in accordance with their usual meaning in the art.

Other abbreviations and definitions may be provided elsewhere in this patent specification, or may be well known in the art.

### Active Agents for Use in the Compositions and Methods of the Invention

As described in the Summary of the Invention and other sections of this patent application, the methods and compositions provided by the present invention involve various different active agents, including, but not limited to, “bi-specific activators,” CD40 agonist s (e.g. CD40 agonist antibodies), TLR agonists, immune checkpoint inhibitors (such as immune checkpoint inhibitor antibodies, PD-1 inhibitors (such as PD-1 inhibitor antibodies), PD-L1 inhibitors (such as PD-L1 inhibitor antibodies), CTLA-4 inhibitors (such as CTLA-4 inhibitor antibodies), and IL10 receptor blocking antibodies. Each of the embodiments described herein that involves one or more of such active agents, such as those known in the art (including, but not limited to the specific exemplary agents described herein), can, in some embodiments, be carried out using any suitable analogues, homologues, variants, or derivatives of such agents. Such analogues, homologues, variants, or derivatives should retain the key functional properties of the specific molecules described herein. For example, in the case of the CD40 agonist antibodies, any suitable analogue, homologue, variant, or derivative of such an antibody can be used provided that it retains CD40 agonist activity. In the case of the TLR agonists, any suitable analogue, homologue, variant, or derivative of such an agent can be used provided that it retains TLR agonist activity. In the case of PD-1 inhibitors, any suitable analogue, homologue, variant, or derivative of such an agent can be used provided that it retains PD-1 inhibitory activity. In the case of PD-L1 inhibitors, any suitable analogue, homologue, variant, or derivative of such an agent can be used provided that it retains PD-L1 inhibitory activity. In the case of CTLA-4 inhibitors, any suitable analogue, homologue, variant, or derivative of such an agent can be used provided that it retains CTLA-4 inhibitory activity. Similarly, in the case of IL10 receptor blocking antibodies, any suitable analogue, homologue, variant, or derivative of such an agent can be used provided that it retains IL10 receptor blocking activity.

Several embodiments of the present invention involve antibodies. As used herein, the term “antibody” encompasses intact polyclonal antibodies, intact monoclonal antibodies, single-domain antibody, nanobody, antibody fragments (such as Fab, Fab′, F(ab′)2, and Fv fragments), single chain Fv (scFv) mutants, multi-specific antibodies such as bi-specific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. In some embodiments the antibody can be an immunoglobulin molecule of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked, or conjugated to other molecules such as toxins, radioisotopes, or any of the other specific molecules recited herein.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FW) residues of a human immunoglobulin are replaced with the corresponding residues in an antibody from a non-human species that has the desired specificity, affinity, and capability.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin whereas all or substantially all of the FR regions are those of a human immunoglobulin consensus sequence. Humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

A “monoclonal antibody” (mAb) refers to a homogeneous antibody population involved in the highly specific recognition and binding of a single antigenic determinant, or epitope. This is in contrast to “polyclonal antibodies” that typically include different antibodies directed against different antigenic determinants. The term “monoclonal antibody” encompasses both intact and full-length monoclonal antibodies, as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

In particular, monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g. radioimmunoassay (RIA); enzyme-linked immunosorbent assay (ELISA)) can then be propagated either in in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid.

Alternatively, monoclonal antibodies can be made using recombinant DNA methods, as described in U.S. Pat. No. 4,816,567. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or antigen-binding fragments thereof of the desired species can be isolated from phage display libraries expressing CDRs of the desired species as described (McCafferty et al., 1990, Nature, 348:552-554; Clackson et al., 1991, Nature, 352:624-628; and Marks et al., 1991, J. Mol. Biol., 222:581-597).

Polyclonal antibodies can be produced by various procedures well known in the art. For example, a host animal such as a rabbit, mouse, rat, etc. can be immunized by injection with an antigen to induce the production of sera containing polyclonal antibodies specific for the antigen. The antigen can include a natural, synthesized, or expressed protein, or a derivative (e.g., fragment) thereof. Various adjuvants may be used to increase the immunological response, depending on the host species, and include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Antibodies can be purified from the host's serum.

### Compositions

In certain embodiments, the present invention provides compositions, such as pharmaceutical compositions. The term “pharmaceutical composition,” as used herein, refers to a composition comprising at least one active agent as described herein (e.g. a bi-specific activator agent), and one or more other components useful in formulating a composition for delivery to a subject, such as diluents, buffers, carriers, stabilizers, dispersing agents, suspending agents, thickening agents, excipients, preservatives, and the like.

Some of the compositions, such as pharmaceutical compositions, described herein comprise two or more of the active agents described herein—such as, for example, a bi-specific activator and an additional active agent. In some of such embodiments the active agents may, optionally, be provided: adsorbed to the surface of alum, or within an emulsion, or within a liposome, or within a micelle, or within a polymeric scaffold, or adsorbed to the surface of, or encapsulated within, a polymeric particle, or within an immunostimulating complex or “iscom,” or within charge-switching synthetic adjuvant particle (cSAP), or within PLGA: poly(lactic-co-glycolic acid) particles, or within other nanoparticles suitable for pharmaceutical administration.

In those embodiments of the present invention that involve nanoparticles, any suitable nanoparticle chemistry or nanoparticle technology known in the art may be used. In some embodiments the nanoparticles may comprise one or more agents selected from the group consisting of mannose, chitosan, manosylated chitosan, protamine, chitosan with protamine, albumin, PLGA, and fucoidan. In some embodiments the nanoparticles may comprise a CD40 agonist (e.g. CD40 agonist antibody) on the surface of the nanoparticle. In some embodiments the nanoparticles may comprise an antibody that binds to a tumor associated antigen on the surface of the nanoparticle. In some embodiments the nanoparticles may comprise an IL10 receptor-blocking antibody on the surface of the nanoparticle. In some embodiments the nanoparticles may comprise a TLR agonist within the nanoparticle. In some embodiments the nanoparticles may comprise an immune checkpoint inhibitor (such as a PD-1 inhibitor or PD-L1 inhibitor or CTLA-4 inhibitor) within the nanoparticle. In some embodiments the nanoparticles may comprise any combination of the above agents on the surface on or within the nanoparticles.

### Methods of Treatment

In certain embodiments the present invention provides methods of treatment. As used herein, the terms “treat,” “treating,” and “treatment” encompass achieving a detectable improvement (such as a statistically significant detectable improvement) in one or more clinical indicators or symptoms associated with a tumor. For example, such terms include, but are not limited to, inhibiting the growth of a tumor (or of tumor cells), reducing the rate of growth of a tumor (or of tumor cells), halting the growth of a tumor (or of tumor cells), causing regression of a tumor (or of tumor cells), reducing the size of a tumor (for example as measured in terms of tumor volume or tumor mass), reducing the grade of a tumor, eliminating a tumor (or tumor cells), preventing, delaying, or slowing recurrence (rebound) of a tumor, improving symptoms associated with tumor, improving survival from a tumor, inhibiting or reducing spreading of a tumor (e.g. metastases), and the like.

The term “tumor” is used herein in accordance with its normal usage in the art and includes a variety of different tumor types. It is expected that the present methods and compositions can be used to treat any solid tumor. Suitable tumors that can be treated using the methods and compositions of the present invention include, but are not limited to, melanomas, lung tumors, colon tumors, prostate tumors, ovarian tumors, sarcomas, and breast tumors, and the various other tumor types mentioned in the present patent specification.

In carrying out the treatment methods described herein, any suitable method or route of administration can be used to deliver the active agents or combinations thereof described herein. In some embodiments systemic administration may be employed, for example, oral or intravenous administration, or any other suitable method or route of systemic administration known in the art. In some embodiments intratumoral delivery may be employed. For example, the active agents described herein may be administered directly into a tumor by local injection, infusion through a catheter placed into the tumor, delivery using an implantable drug delivery device inserted into a tumor, or any other means known in the art for direct delivery of an agent to a tumor.

As used herein the terms “effective amount” or “therapeutically effective amount” refer to an amount of an active agent as described herein that is sufficient to achieve, or contribute towards achieving, one or more desirable clinical outcomes, such as those described in the “treatment” description above. An appropriate “effective” amount in any individual case may be determined using standard techniques known in the art, such as dose escalation studies, and may be determined taking into account such factors as the desired route of administration (e.g. systemic vs. intratumoral), desired frequency of dosing, etc. Furthermore, an “effective amount” may be determined in the context of any co-administration method to be used. One of skill in the art can readily perform such dosing studies (whether using single agents or combinations of agents) to determine appropriate doses to use, for example using assays such as those described in the Examples section of this patent application—which involve administration of the agents described herein to subjects (such as animal subjects routinely used in the pharmaceutical sciences for performing dosing studies).

For example, in some embodiments the dose of an active agent of the invention may be calculated based on studies in humans or other mammals carried out to determine efficacy and/or effective amounts of the active agent. The dose amount and frequency or timing of administration may be determined by methods known in the art and may depend on factors such as pharmaceutical form of the active agent, route of administration, whether only one active agent is used or multiple active agents (for example, the dosage of a first active agent required may be lower when such agent is used in combination with a second active agent), and patient characteristics including age, body weight or the presence of any medical conditions affecting drug metabolism.

In those embodiments described herein that refer to specific doses of agents to be administered based on mouse studies, one of skill in the art can readily determine comparable doses for human studies based on the mouse doses, for example using the types of dosing studies and calculations described herein.

In some embodiments suitable doses of the various active agents described herein can be determined by performing dosing studies of the type that are standard in the art, such as dose escalation studies, for example using the dosages shown to be effective in mice in the Examples section of this patent application as a starting point. Interestingly, and as illustrated in the Examples, it has been found that the methods and compositions of the present invention are effective using much lower doses of the active agents than would normally be used in other applications and contexts. In some embodiments, where the active agents used are antibodies, the agents are administered at a dose of from about 1 mg/kg to about 10 mg/kg, or at a dose of from about 0.1 mg/kg to about 10 mg/kg.

Dosing regimens can also be adjusted and optimized by performing studies of the type that are standard in the art, for example using the dosing regimens shown to be effective in mice in the Examples section of this patent application as a starting point. In some embodiments the active agents are administered daily, or twice per week, or weekly, or every two weeks, or monthly.

In certain embodiments the compositions and methods of treatment provided herein may be employed together with other compositions and treatment methods known to be useful for tumor therapy, including, but not limited to, surgical methods (e.g. for tumor resection), radiation therapy methods, treatment with chemotherapeutic agents, treatment with antiangiogenic agents, or treatment with tyrosine kinase inhibitors. Similarly, in certain embodiments the methods of treatment provided herein may be employed together with procedures used to monitor disease status/progression, such as biopsy methods and diagnostic methods (e.g. MRI methods or other imaging methods).

For example, in some embodiments the agents and compositions described herein may be administered to a subject prior to performing surgical resection of a tumor, for example in order to shrink a tumor prior to surgical resection. In other embodiments the agents and compositions described herein may be administered both before and after performing surgical resection of a tumor. In other embodiments the subject has no tumor recurrence after the surgical resection.

### Subjects

As used herein the term “subject” encompasses all mammalian species, including, but not limited to, humans, non-human primates, dogs, cats, rodents (such as rats, mice and guinea pigs), cows, pigs, sheep, goats, horses, and the like—including all mammalian animal species used in animal husbandry, as well as animals kept as pets and in zoos, etc. In preferred embodiments the subjects are human. Such subjects will typically have (or previously had) a tumor (or tumors) in need of treatment. In some embodiments the subject has previously been treated with an immune checkpoint inhibitor (such as a PD-1 inhibitor, PD-L1 inhibitor, or a CTLA-4 inhibitor). In some embodiments the subject has not previously been treated with an immune checkpoint inhibitor (such as a PD-1 inhibitor, PD-L1 inhibitor, or a CTLA-4 inhibitor). In some embodiments the subject has a tumor that is insensitive to, or resistant to, treatment with an immune checkpoint inhibitor (such as a PD-1 inhibitor, PD-L1 inhibitor, or a CTLA-4 inhibitor), or that is suspected of being insensitive to, or resistant to, treatment with an immune checkpoint inhibitor (such as a PD-1 inhibitor, PD-L1 inhibitor, or a CTLA-4 inhibitor). In some embodiments the subject has a tumor that has recurred following a prior treatment with an immune checkpoint inhibitor (such as a PD-1 inhibitor, PD-L1 inhibitor, or a CTLA-4 inhibitor) and/or with one or more other tumor treatment methods, including, but not limited to, chemotherapy, radiation therapy, or surgical resection, or any combination thereof. In some embodiments the subject has a tumor that has not previously been treated, whether with an immune checkpoint inhibitor (such as a PD-1 inhibitor, PD-L1 inhibitor, or a CTLA-4 inhibitor) or with one or more other tumor treatment methods, including, but not limited to, chemotherapy, radiation therapy, or surgical resection, or any combination thereof.

### EXAMPLES

The invention is further described in the following non-limiting Examples, as well as the Figures referred to therein.

### Example 1

### Mouse Bi-Lateral Tumor Model

Immune checkpoint blockade (for example using anti-CTLA-4, PD-1, and PD-L1 monoclonal antibodies (mAbs)) offers the potential for durable remissions for patients across a broad range of cancers, including, but not limited to, lung, breast, colon and prostate cancer. However, despite this broad applicability, the majority (well over 80%) of cancer patients are, or become, resistant to it. The studies presented in this Example demonstrate an approach to overcome resistance to immune checkpoint blockade in manner applicable to most cancers, regardless of type or stage.

Cancers refractory to immune checkpoint blockade generally fail to mount significant antitumor T lymphocyte responses. Many cancers, including breast and colon cancer demonstrate defective antigen presenting cell (APC) activation. Since APCs prime T lymphocytes, this can explain the absence of a productive anti-tumor T lymphocyte response in these cancers.

Various active agents, or combinations, thereof, were tested in a murine model of aggressive melanoma, shown to be resistant to checkpoint blockade, with the aim of testing this hypothesis and identifying treatments with potent anti-tumor activity. The animals used had established tumors in two opposite flanks. One tumor was injected while the second remained non-injected (FIG. 1), allowing separate analysis of the effect at the injected tumor from the so-called ‘abscopal’ effect at the non-injected tumor, in order to understand how this treatment could benefit patients with metastatic cancer. However, in clinical applications multiple tumor sites can be injected. To test whether resistance to anti-PD-1 therapy can be reversed, we used the poorly immunogenic B16 murine melanoma model previously shown to be relatively resistant to PD-1 blockade. C57BL/6 mice were initially implanted with 5×10⁵ syngeneic B16F10 cells intra-dermally in bilateral flanks. 8 days post tumor cell implantation, when bilateral tumors measured ˜0.5 cm in diameter, intratumoral (IT) treatment with various test and control agents was initiated together with, in some instances, intraperitoneal (IP) anti-PD-1 mAb. Treatment was administered twice weekly for 4 weeks into one of the bilateral tumors. The contralateral tumor remained un-injected for the duration of the experiment. FIG. 1 provides a schematic illustration of this experimental protocol.

### Example 2

### Bi-Specific Activators Comprising an Anti-TA99 Antibody

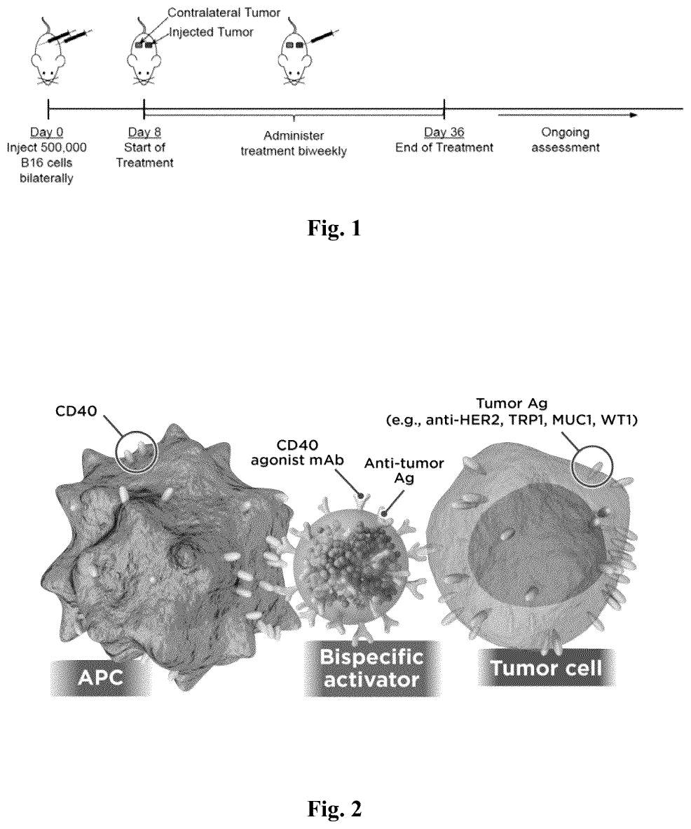

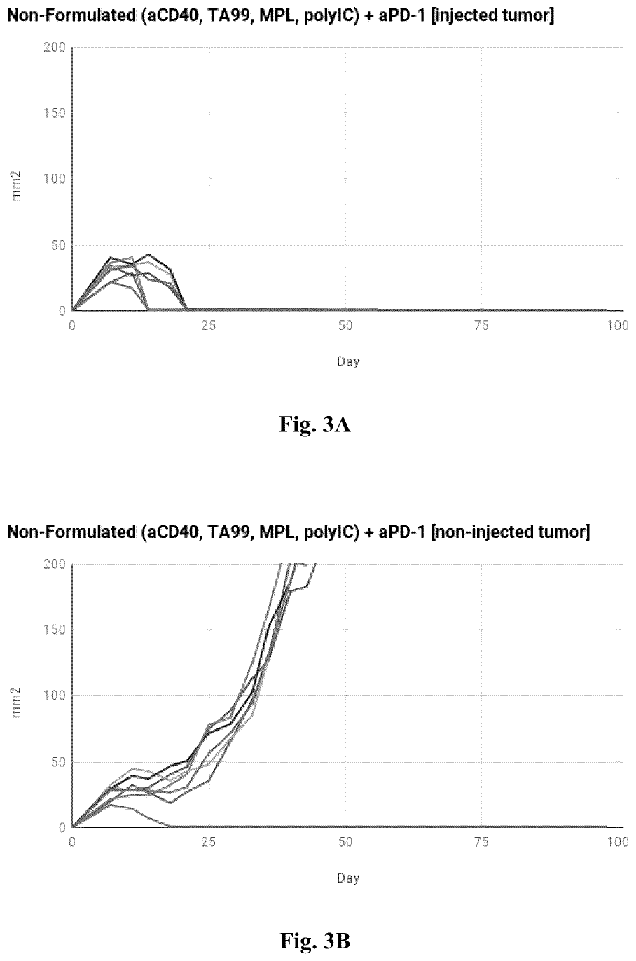

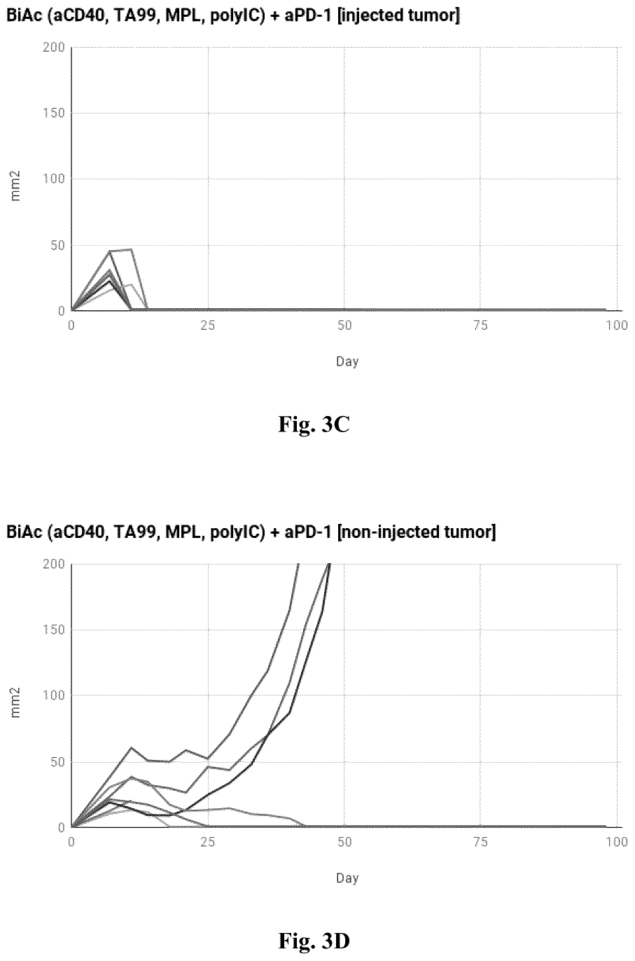

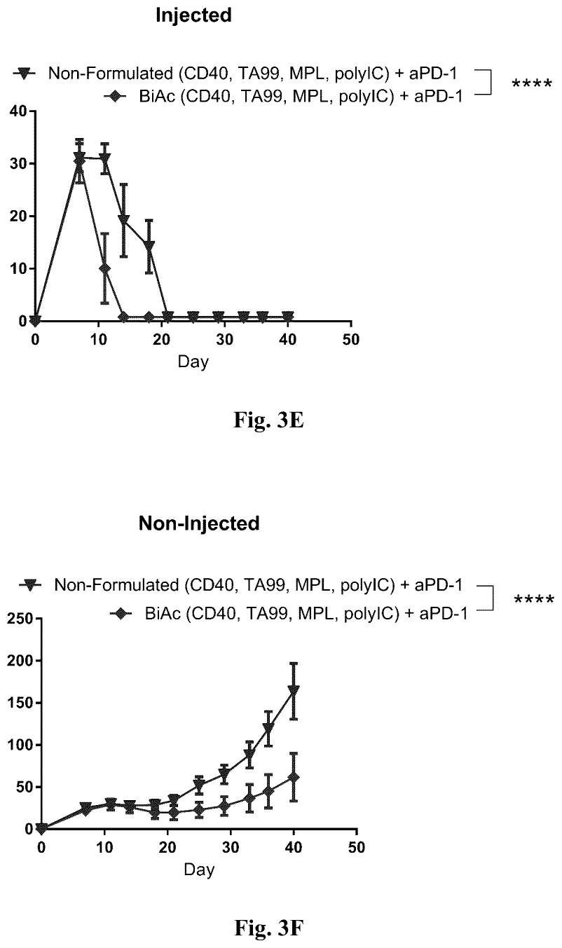

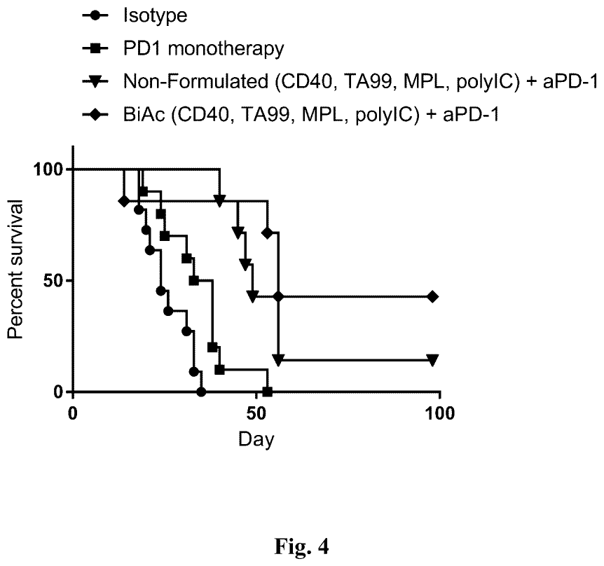

Experiments were performed to test the effects of an exemplary “bi-specific activator” nanoparticle of the type illustrated schematically in FIG. 2. “Test” of “formulated” treatment groups were treated with bi-specific activator nanoparticles having a CD40 agonist antibody (FGK45) and an antibody (TA99) specific for the tumor-associated antigen TRP1 on the surface of the chitosan nanoparticles, and an internal cargo of MPL and poly:IC. “Control” or “non-formulated” treatment groups were treated with the same agents 4 agents (TA99, FGK45, MPL, and polyIC) at concentrations equivalent to those in the test group above were delivered as a mixture in PBS without a nanoparticle. The control and test treatments were administered to C57BL/6 animals bearing bilateral intradermal flank B16 (melanoma) tumors as depicted in FIG. 1. Treatments were administered twice weekly to one of the two established tumors. All animals (in both the control and experimental group) also received concurrent (twice weekly) PD-1 blocking mAb RMP1-14. Each intratumoral treatment (i.e., injection) contained 5 micrograms of MPL, 20 micrograms of FGK45, 20 micrograms of TA99, and 10 micrograms of polyIC. These agents were physically associated with a 120 nm chitosan nanoparticle in the experimental group but not in the control group. Animals in both the treatment and control groups received 250 micrograms of anti-PD-1 mAb RMP1-14 intraperitoneally on the same days as the intratumoral treatments. Results from these experiments are shown in FIGS. 3A-F and FIG. 4. The bi-specific activator provided superior tumor control both locally and systemically as compared to the non-formulated composition, fully regressed injected tumors faster than the non-formulated mixture, and regressed/delayed tumor growth more effectively at the contralateral tumor (FIG. 3A-F). The bi-specific activator also increased survival times as compared to the non-formulated composition (FIG. 4).

### Example 3

### Additional Bi-Specific Activators

While the experiments described in Example 2 involved bi-specific nanoparticles comprising an antibody to the melanoma associated antigen TRP1, antibodies to a variety of tumor-associated antigens can be used. Similarly, several of the specific components of the bi-specific nanoparticles described in Example 2 can be adjusted.

Bi-specific activators can be made comprising a CD40 agonist antibody (e.g. FGK45) and an antibody specific for a tumor-associated antigen (e.g. an antibody to tyrosinase, gp100/pmel, Melan-A/MART-1, TRP1, or TRP2) on the surface of nanoparticles (such as nanoparticles comprising mannose, chitosan, manosylated chitosan, protamine, chitosan with protamine, albumin, PLGA, and/or fucoidan). One or more TLR agonists (e.g. MPL and/or poly:IC) can optionally be included within the nanoparticles as “cargo.”

Experiments can be performed follows: In “control” or “non-formulated” treatment groups the antibody to the melanoma antigen, the CD40 agonist antibody, and, if present, the one or more TLR agonists, each at concentrations equivalent to those used above in Example 2, can be delivered as a mixture in PBS without a nanoparticle. In “test” or “formulated” treatment groups, the antibody to the melanoma antigen, the CD40 agonist antibody, and, if present, the one or more TLR agonists, can be physically associated with a nanoparticle, as in Example 2. Test and control treatments can be administered to C57BL/6 animals bearing bilateral intradermal flank B16 (melanoma) tumors as depicted in FIG. 1. The treatments may be administered twice weekly to one of the two established tumors. Animals (in both control and experimental groups) can also receive concurrently (e.g. twice weekly) an immune checkpoint inhibitor, (such as the PD-1 blocking mAb RMP1-14). Each intratumoral treatment (i.e., injection) can contain 5 micrograms of MPL, 20 micrograms the CD40 agonist antibody, 20 micrograms of the antibody to the melanoma antigen, and 10 micrograms of polyIC. If an immune checkpoint inhibitor is also administered, animals in both groups can also receive 250 micrograms of the immune checkpoint inhibitor (such as the anti-PD-1 mAb RMP1-14) intraperitoneally, for example on the same days as the intratumoral treatments with the other agents. It is expected that in the “test” groups the bi-specific activator compositions can provide superior tumor control (both locally and systemically) as compared to that in the “control” groups treated with the non-formulated mixtures of components.

1-72. (canceled)

73. A method of treating a tumor, the method comprising administering to a subject in need thereof an effective amount of a composition comprising nanoparticles, wherein the nanoparticles comprise both (a) CD40 agonist antibody and (b) an antibody specific for a tumor-associated antigen (TAA) on their surface, and also comprise a pro-inflammatory agent or a vaccine adjuvant.

74. The method of claim 73, wherein the nanoparticles also comprise a TLR agonist inside the nanoparticle.

75. The method of claim 73, wherein the CD40 agonist antibody is selected from the group consisting of FGK45, CP-870,984, CP-870,983, APX005M, dacetuzumab, and ChiLob 7/4.

76. The method of claim 73, wherein the TAA is selected from the group consisting of TRP1, her2, muc1, muc16, CD19, CD20, CD38, SLAMF7, and EGFRvIII.

77. The method of claim 73, wherein the TAA is a melanoma-associated antigen is selected from the group consisting of tyrosinase, gp100/pmel, Melan-A/MART-1, gp75/TRP1, and TRP2.

78. The method of claim 74, wherein the TLR agonist binds to a TLR expressed by antigen presenting cells (APCs) selected from the group consisting of dendritic cells (DCs), macrophages, tissue-resident macrophages, monocytes, monocyte-derived cells, B-Cells, neutrophils, langerhans cells, histiocytes, professional APCs, and non-professional APCs.

79. The method of claim 74, wherein the TLR agonist is a TLR4 agonist or a TLR3 agonist.

80. The method of claim 79, wherein the TLR4 agonist is monophosphoryl lipid A (MPL) or the TLR3 agonist is polyI:C.

81. The method of claim 1, wherein the nanoparticles comprise one or more agents selected from the group consisting of mannose, chitosan, manosylated chitosan, protamine, chitosan with protamine, albumin, PLGA, and fucoidan.

82. The method of claim 73, wherein the nanoparticles further comprise an IL10 receptor blocking antibody or an IL10 blocking antibody on their surface.

83. The method of claim 73, wherein the composition is administered locally, such as intratumorally.

84. A composition comprising nanoparticles, wherein the nanoparticles comprise both (a) a CD40 agonist antibody and (b) an antibody specific for a tumor associated antigen (TAA) on their surface, and wherein the nanoparticles also comprise a TLR agonist inside the nanoparticles.

85. The composition of claim 84, wherein the CD40 agonist antibody is selected from the group consisting of FGK45, CP-870,984, CP-870,983, APX005M, dacetuzumab, and ChiLob 7/4.

86. The composition of claim 84, wherein the TAA is selected from the group consisting of TRP1, her2, muc1, muc16, CD19, CD20, CD38, SLAMF7, EGFRvIII tyrosinase, gp100/pmel, Melan-A/MART-1, gp75/TRP1, and TRP2.

87. The composition of claim 84, wherein the TAA is a melanoma-associated antigen selected from the group consisting of tyrosinase, gp100/pmel, Melan-A/MART-1, gp75/TRP1, and TRP2

88. The composition of claim 84, wherein the TLR agonist is a TLR4 agonist or a TLR3 agonist.

89. The composition of clam 84, wherein the TLR agonist is monophosphoryl lipid A (MPL).

90. The composition of clam 84, wherein the TLR agonist is polyI:C.

91. The composition of claim 84, wherein the nanoparticles comprise one or more agents selected from the group consisting of mannose, chitosan, manosylated chitosan, protamine, chitosan with protamine, albumin, PLGA, and fucoidan.

92. The composition of claim 84, wherein the nanoparticles further comprise an IL10 receptor blocking antibody or an IL10 blocking antibody on their surface.
